(19) United States
(12) Patent Application Publication
(10) Pub. No.: US 2016/0015661 A1
(43) Pub. Date: Jan. 21, 2016

(54) ASSEMBLY COMPRISING AN ABSORBER OF NEAR INFRARED (NIR) LIGHT COVALENTLY LINKED TO AN INHIBITOR OF CARBONIC ANHYDRASE

(71) Applicant: CONSIGLIO NAZIONALE DELLE RICERCHE, Rome (IT)

(72) Inventors: Roberto PINI, Impruneta (FIRENZE) (IT); Fulvio RATTO, Campi Bisenzio (FIRENZE) (IT); Francesca TATINI, San Giovanni Valdarno (AREZZO) (IT); Franco FUSI, ROMA (IT); Sonia CENTI, Capraia Fiorentina (FIRENZE) (IT); Andrea SCOZZAFAVA, Bagno a Ripoli (FIRENZE) (IT); Claudiu Trandafir SUPURAN, FIRENZE (IT); Fabrizio CARTA, Irgoli (NUORO) (IT); Sergio CAPACCIOLI, FIRENZE (IT); Ewa Janina WITORT, Figline e Incisa Valdarno (FIRENZE) (IT)

(73) Assignee: CONSIGLIO NAZIONALE DELLE RICERCHE, ROMA (IT)

(21) Appl. No.: 14/773,046

(22) PCT Filed: Mar. 6, 2014
(86) PCT No.: PCT/IB2014/059490

§ 371 Date: Sep. 4, 2015

(30) Foreign Application Priority Data
Mar. 7, 2013 (IT) RM2013A000138

(51) Int. Cl. A61K 31/185; A61N 5/06; A61K 49/22; A61K 47/48; A61K 41/00

(52) U.S. Cl. CPC A61K 31/185; A61K 47/48884; A61K 41/0052; A61K 49/225; A61K 49/221; A61N 5/0625; A61N 5/062

(57) ABSTRACT

An assembly comprising an absorber of near infrared (NIR) light having an optical absorption cross section not lower than 100 nm2 covalently linked to an inhibitor of carbonic anhydrase (CA), process for its preparation and its use to hyperthermally target tumours or for treating other conditions in which the CA activity is involved is disclosed.

### BACKGROUND OF THE INVENTION

The present invention refers to the pharmaceutical field and in particular to an assembly comprising an absorber of near infrared (NIR) light covalently linked to an inhibitor of carbonic anhydrase (CA) specifically designed to hyperthermally target tumours by application of a near infrared light, for example from a laser, or for treating other conditions in which the CA activity is involved.

### PRIOR ART

It is known in the art the use of absorbers of near infrared (NIR) light for photo-acoustic imaging and optical hyperthermia of cancer.

Furthermore, combinations of absorbers of near infrared (NIR) light with ligands are known in the art. The ligands are intended to exploit constitutive anomalies of a specific subpopulation of malignant cells, which may vary from lesion to lesion and provide poor specificity. The variability underlies the complexity of cancer. A deficit of specificity mirrors the lack of qualitative differences between normal and malignant cells, which often exhibit the same biochemical diversity but at different levels of expression. Examples include the overexpression of epidermal growth factor receptors, vascular endothelial growth factor receptors, folate receptors, etc., which reflects the higher metabolic activity of malignant cells. Although with a different density these species are common to both normal and malignant cells.

Wang M. and Thanou M., 2010, “Targeting nanoparticles to cancer”, Pharmacological Research 62, 90-99 reviews nanotechnology applications in medicine, wherein devices with dimensions on the nanometer length scale can carry detection signals and/or therapeutic cargos and are designed to reach their target and specifically release their load, thus enhancing the drug's tissue bioavailability. They comprise liposomes, solid lipid nanoparticles (SLN), dendrimers, iron oxide nanoparticles used for imaging purposes, noble metal nanoparticles such as gold, noble metal nanoshells consisting of a dielectric bead surrounded by a thin metal shell, such as gold. For example, NIR laser treatment of the bulk tissue selectively heats and destroys the nanoshell-laden tumor tissue, while leaving surrounding tissue intact, or nanorods with the appropriate PEG stealth layer are being developed as an improved means of optical hyperthermia.

Huang X. et al., 2010, “A Reexamination of Active and Passive Tumor Targeting by Using Rod-Shaped Gold Nanocrystals and Covalently Conjugated Peptide Ligands”, 2010, ACSNANO 4, 5887-5896, discloses colloidal gold nanorods stabilized with polyethylene glycols (PEGs) conjugated to three different ligands: a single-chain variable fragment (ScFv) peptide that recognizes the epidermal growth factor receptor (EGFR); an amino terminal fragment (ATF) peptide that recognizes the urokinase plasminogen activator receptor (uPAR); a cyclic RGD peptide that recognizes the aᵥβ₃ integrin receptor. Quantitative tumour uptake studies show that the use of active targeting ligands only marginally improves the total tumor uptake in comparison with control particles resting on the enhanced permeability and retention (EPR) pathway only. However, the nature of targeting ligands can affect the nanoparticle distribution in the tumor cells and the tumor microenvironments, suggesting that for photothermal cancer therapy, the preferred route of gold nanorods is intratumoral administration.

The use of inhibitors of carbonic anhydrases as therapeutic agents has been reported for the management of a variety of disorders (glaucoma, edema, obesity, cancer, epilepsy, etc. (Supuran, Nature Rev. Drug. Discov. 7, 168-181 (2008)).

Carbonic anhydrases exist in several isoforms and are ubiquitous within eukaryotic cells. However, Carbonic anhydrase 9 (CA9) is a transmembrane protein which is overexpressed by cells only under conditions of deficient oxygenation. Therefore an inhibitor of CA9 which may not penetrate plasmatic membranes would specifically bind hypoxic cells (Stiti, J. Am. Chem. Soc. 130, 16130-16131 (2008)). Hypoxia occurs in a broad variety of solid tumors as a result of their abnormal growth and insufficient vascularization. As a rule solid tumors feature a coexistence of necrotic, hypoxic and normoxic tissue. Furthermore, it is widely recognized that cancer is maintained by a small population of cancer stem cells, which are able to indefinitely divide, exhibit resistance to chemotherapeutics and require a hypoxic environment.

WO2004048544 discloses aromatic sulphonamide and heterocyclic sulfonamide CA9 specific inhibitors.

U.S. 20070212305 discloses optical imaging techniques of lung cancer by means of a contrast agent linked with a ligand for an overexpressed target associated with lung cancer. Among the targets it is mentioned carbonic anhydrase.

U.S. 20090104179 discloses a luminescent material responsive to ionizing radiation and a photosensitive biologically active material.

U.S. 20110262500 discloses nanoparticles of gold, silver or platinum bound by means of a linker to a platinum compound for cancer treatment.

U.S. 20130004523 discloses paclitaxel covalently linked to a nanoparticle.

WO9725039 discloses the combination between carbonic anhydrase inhibitors and another agent for multimodal treatment of cancer.

WO2007046893 discloses a dock and lock method to assemble effector moieties for treating cancer. Among the effector moieties of interest are mentioned ligands and anticancer agents such as those for optical hyperthermia.

WO2012018383 discloses gold nanoparticles conjugates with thiloated hyperbranched dendrons with moieties capable to carry anticancer agents.

WO2006137009 and WO2006137092 disclose CA9 inhibitors comprising a moiety of a fluorescent dye.

WO2008071421 discloses nitrate ester derivatives of carbonic anhydrase inhibitors.

Further classes of CA9 inhibitors are disclosed in WO201109861, WO2012021963, WO2012070024 and WO2012175654.

U.S. 20120321563 discloses fluorescent imaging agents that bind to carbonic anhydrase, in particular CA9, used for identifying hypoxic cells in hypoxic tumors. In particular said agents comprise a far-red or near-infrared fluorophore linked to a carbonic anhydrase targeting agent. The fluorophore is a chemical such as a cyanine dye, carbocyanine dye, indocyanine dye, or a polymethine fluorescent dye providing a fluorescent signal detectable by imaging techniques.

WO2012154885 discloses carbonic anhydrase targeting agents chemically linked to fluorophores in the far-red or near-infrared region or radiolabels, optionally by a linker, which can be used to image or deliver radiation to hypoxic tumors.

Ratto F. et al., 2011, “Gold nanorods as new nanochromophores for photothermal therapies”, Journal of Biophotonics 4, 64-73, disclose the use of colloidal gold nanorods excited by diode laser radiation at 810 nm to mediate functional photothermal effects, in particular laser welding mediated by aqueous colloids of gold nanorods or gold nanorods into biopolymeric solders. The authors also speculate the possible use of gold nanorods conjugated with antibodies, aptamers, natural factors, peptides, etc. in diagnosis, imaging and therapy of cancer.

Stiti M. et al., 2008, “Carbonic anhydrase inhibitor coated gold nanoparticles selectively inhibit the tumor-associated isoform IX over the cytosolic isozymes I and II”, J. Am. Chem. Soc. 130, 16130-16131 disclose gold nanoparticles coated with sulphonamide carbonic anhydrase inhibitors which can be used for targeting tumors overexpressing CA9. Said gold nanoparticles are not near infrared absorbers.

The compounds disclosed, roughly spherical in shape, of empirical formulas [Au₇₂₀ (C₁₆H₂₄O₃N₂S₃)₁₄₄] and [Au₇₂₄ (C₁₇H₂₁O₃N₃S₄)₁₃₅], have an average particle size of 3.3 nm which corresponds to ˜720-724 Au atoms and a number of sulfonamide ligands attached to the NP as being 144 and 135 respectively.

[Au₇₂₀ (C₁₆H₂₄O₃N₂S₃)₁₄₄] shows excellent CA9 inhibitory properties and selectivity for the inhibition of the tumor-associated isoform over carbonic anhydrases 1 and 2, while [Au₇₂₄(C₁₇H₂₁O₃N₃S₄)₁₃₅] has been used as a control, since substituted sulfonamides do not act as CAIs.

Groves et al., 2011, Bioorganic & Medicinal Chemistry Letters, 22(1), 653:657 disclose human carbonic anhydrase (hCA) IX inhibitors conjugated to fluorescent dyes with the aim of imaging hypoxia-induced hCA IX expression in tumour cells.

Tafreshi et al., 2012, Clinical Cancer Research, 18(1), 207:219 disclose carbonic anhydrase IX-specific monoclonal antibodies conjugated to a near-infrared fluorescent dye as molecular imaging probes.

Maamar Stiti et al., 2008, Journal of the American Chemical Society, 130(48), 16130:16131 disclose gold nanoparticles coated with sulfonamide carbonic anhydrase inhibitors.

Ratto et al., 2009, Journal of Nanoparticle Research, 12(6), 2029:2036 report the control of the overgrowth of gold nanorods by modulating their size and shape.

The distinguishing feature of the present invention is the new covalent combination between an inhibitor of carbonic anhydrase and an absorber of near infrared (NIR) light within the assembly of the present invention, wherein the absorber of near infrared (NIR) light has an optical absorption cross section not lower than 100 nm².

The optical absorption cross section of the single unit of absorber of near infrared (NIR) light needs to exceed 100 nm² because the concentration of CA9 in hypoxic lesions is normally not higher than 10¹²-10¹³ molecules per ml (G. X. Zhou, J. Ireland, P. Rayman, J. Finke, M. Zhou, “Quantification of carbonic anhydrase IX expression in serum and tissue of renal cell carcinoma patients using enzyme-linked immunosorbent assay: prognostic and diagnostic potentials,” Urology. 75, 257-61 (2010); M. Takacova, M. Bartosova, L. Skvarkova, M. Zatovicova, I. Vidlickova, L. Csaderova, M. Barathova, J. Breza, P. Bujdak, J. Pastorek, J. Breza, S. Pastorekova, “Carbonic anhydrase IX is a clinically significant tissue and serum biomarker associated with renal cell carcinoma,” Oncol Lett. 5, 191-7 (2013)) and the optical absorption coefficient needed for optical hyperthermia is in the order of 1-10 cm⁻¹ (i.e. higher than that typical of endogenous tissue, see J. Mobley, T. Vo-Dinh, “Optical properties of tissue,” 2-60-70 in T. Vo-Dinh Ed., “Biomedical photonics handbook,” CRC Press, Boca Raton (2003)).

Said combination shows a synergistic effect in improving efficacy and specificity of hyperthermia therapy.

On optical stimulation, the assembly of the present invention acts as anticancer agent in hyperthermia therapy.

The technical problem of the present invention is to provide anticancer agents different from those disclosed in the prior art having better properties when used in hyperthermia therapy.

The combination between an inhibitor of carbonic anhydrase and an absorber of near infrared (NIR) light with an optical absorption cross section not lower than 100 nm², which is inert without optical activation, allows to specifically target malignant lesions.

Moreover, the site-directed optical activation of the absorber of near infrared (NIR) light with an optical absorption cross section not lower than 100 nm² by local illumination of the tumour avoids the risk of damaging hypoxic benign lesions as well as those organs where the assembly may accumulate after systemic administration, such as liver and spleen.

Moreover, said controlled optical activation also extends the effect of hyperthermia, primarily directed to the hypoxic core of the malignant lesion, to its adjacent normoxic periphery, thanks to heat dissipation.

The assembly between an inhibitor of carbonic anhydrase and an absorber of near infrared (NIR) light with an optical absorption cross section not lower than 100 nm² enhances specificity and versatility.

Specificity arises from the high contrast in terms of expression of transmembrane carbonic anhydrase between normoxic and hypoxic tissue, while versatility originates from the ubiquity of carbonic anhydrase 9 expression under hypoxic conditions.

The assembly of the present invention provides synergistic opportunities since both carbonic anhydrase targeting and optical hyperthermia profit from blood stagnation in solid tumours, which arouses hypoxia and jeopardizes the principal pathways of heat dissipation.

### OBJECT OF THE INVENTION

As already discussed, the technical problem has been solved by providing an assembly comprising an inhibitor of carbonic anhydrase and an absorber of near infrared (NIR) light covalently bound by a linker wherein the absorber of near infrared (NIR) light has an optical absorption cross section not lower than 100 nm², which is the object of the present invention.

Another object of the present invention is said assembly optionally further comprising at least one drug or oligonucleotide bound to the absorber of near infrared (NIR) light by a cross-linker able to dissociate at a temperature between 40-100° C.

Another object of the present invention is the use of said assembly as a contrast agent for photothermal treatments.

The assembly of the present invention may also be used as a contrast agent for photoacoustic imaging.

A further object of the present invention is the use of said assembly as a medicament, in particular as an anticancer agent in hyperthermia therapy of tumours or conditions in which the CA activity is involved.

It is an object of the present invention a process for the preparation of said assembly comprising the following steps:
a) Contacting the NIR absorber with the linker and reacting to obtain a covalent bond between the NIR absorber and the linker;
b) Contacting the NIR absorber covalently bound to the linker as obtained in step a) with the CAI and reacting to obtain the assembly.

Or alternatively:
a) Contacting the CAI with the linker and reacting to obtain a covalent bond between the CAI and the linker;
b.) Contacting the CAI covalently bound to the linker as obtained in step a) with the NIR absorber and reacting to obtain the assembly.

Further features of the present invention will be clear from the following detailed description with references to experimental examples and attached figures.

### BRIEF DESCRIPTION OF THE FIGURES

FIG. 1 shows a schematic representation of one object of this invention.

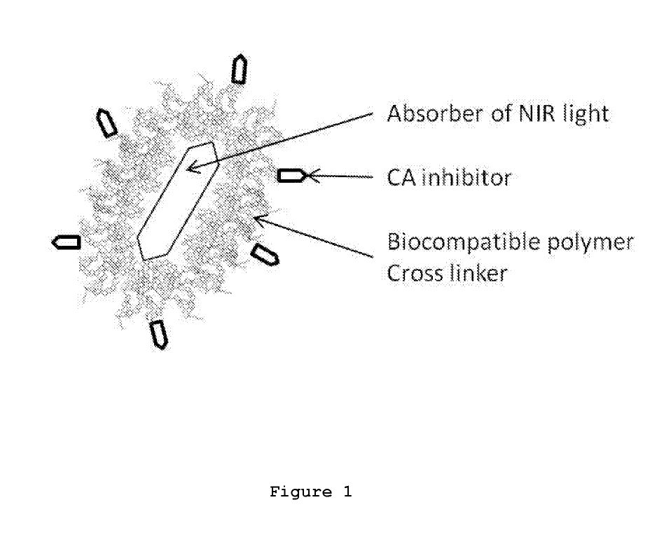

FIG. 2 shows the targeting of gold particles with inhibitors of CA9. Images display HCT116 colon carcinoma cells expressing transmembrane carbonic anhydrases only under hypoxic conditions. The accumulation of gold particles is revealed by silver enhancement only in the presence of the carbonic anhydrase inhibitor and under hypoxia.

FIG. 3 shows images of HCT116 colon carcinoma cells kept under hypoxic conditions and then treated for ten minutes with a NIR laser with a power density of 60 W cm². Panels display cells without particles (A) and incubated with PEGylated particles without (B) and with ligand (C). After incubation, the growth medium was thoroughly replaced. The synergistic effect of the inhibitor and optical excitation leads to cell death, which is identified by trypan blue staining in panel C. In the absence of ligand this optical fluence does not damage cells. The power density required to reach the damage threshold both under hypoxia with particles without ligands and normoxia with particles with ligands falls between around 120-150 W cm⁻².

### DETAILED DESCRIPTION OF THE INVENTION

The main object of the present invention is an assembly comprising an absorber of near infrared (NIR) light and an inhibitor of carbonic anhydrase covalently bound by a linker wherein the absorber of near infrared (NIR) light has an optical absorption cross section not lower than 100 nm².

The above absorber of near infrared (NIR) light is not a single fluorescent molecule since the value of optical absorption cross section for single fluorescent molecules is in the order of 0.01 nm² (e.g. about 0.02 nm² for indocyanine green in water, which is among the most efficient near infrared dyes).

The above values are calculated considering the following parameters: optical hyperthermia requires an optical absorption coefficient (a) around 1-10 cm ⁻¹ in order to achieve contrast with endogenous tissue (see J. Mobley, T. Vo-Dinh, “Optical properties of tissue,” 2-60-70 in T. Vo-Dinh Ed., “Biomedical photonics handbook,” CRC Press, Boca Raton (2003)) and the concentration of CA9 in hypoxic tumors is normally below 10¹²-10¹³ molecules per ml (G. X. Zhou, J. Ireland, P. Rayman, J. Finke, M. Zhou, “Quantification of carbonic anhydrase IX expression in serum and tissue of renal cell carcinoma patients using enzyme-linked immunosorbent assay: prognostic and diagnostic potentials,” Urology. 75, 257-61 (2010); M. Takacova, M. Bartosova, L. Skvarkova, M. Zatovicova, I. Vidlickova, L. Csaderova, M. Barathova, J. Breza, P. Bujdak, J. Pastorek, J. Breza, S. Pastorekova, “Carbonic anhydrase IX is a clinically significant tissue and serum biomarker associated with renal cell carcinoma,” Oncol Lett. 5, 191-7 (2013)), which represents an upper limit for the density (d) of absorbers of near infrared (NIR) light that may be accumulated in hypoxic tumors by the use of this molecular target. A lower limit for the optical absorption cross section (a) of the single unit of absorber of near infrared (NIR) light is given by the formula σ=α/d.

Within the meaning of the present invention assembly means a combination of elements covalently linked to each other.

Within the meaning of the present invention plasmonic particles means particles whose electron density can couple with electromagnetic radiation of wavelengths that are larger than the particle size due to the nature of the dielectric-metal interface between the medium and the particles.

Within the meaning of the present invention absorber of near infrared (NIR) light means any compound capable to mediate photothermal conversion at NIR frequencies and having an optical absorption cross section not lower than 100 nm². Examples include nanoparticles containing near infrared dyes (organic compounds with conjugated n systems containing polyene, polymethine and donor-acceptor near infrared chromophores, including indocyanine green, IR-780 iodide) such as solid lipid, liposomal, micellar, porous silica, polymeric, viral carriers; phospholipid porphyrin conjugates; organic nanoparticles such as single walled and multi walled carbon nanotubes, graphene, graphene oxide; metallic nanoparticles with near infrared plasmonic resonances such as gold nanorods, gold/silica nanoshells, gold nanocages, gold/gold sulfide nanoshells, hollow gold nanospheres; upconversion nanoparticles containing lanthanide moieties, and combinations thereof.

Preferably the absorber of near infrared (NIR) light has an optical absorption cross section ranging from 10² to 10⁵ nm².

More preferably the absorber of near infrared (NIR) light has an optical absorption cross section ranging from 10³ to 10⁴ nm².

Preferably the absorber of near infrared (NIR) light is a plasmonic particle in a range of size between ˜5 and 100 nm.

Preferably the absorber of near infrared (NIR) light is a gold nanorod.

Preferably the absorber of near infrared (NIR) light is impermeable to plasmatic membranes in order to inhibit its penetration inside the cellular cytoplasm and prevent its association with intracellular carbonic anhydrases isoforms.

Within the meaning of the present invention inhibitor of carbonic anhydrase means any compound able to inhibit the activity of carbonic anhydrase by specific binding.

Preferably the inhibitor of carbonic anhydrase is an inhibitor of isoform carbonic anhydrase 9 (CA9).

Preferably the inhibitor of carbonic anhydrase is the compound of general formula (I):

X-(CH₂)ₙ-Ar-[Q]-SO₂NH₂ (I)
wherein
n is an integer from 0 to 2
Q is absent or is O or N
X is N, O, S or COOR
R is H, Me, Et, n-Pr, halogen

Wherein halogen means Cl, F, I or Br

Ar is an aromatic or heteroaromatic ring optionally substituted with at least one substituent selected from the group consisting of: Cl, F, I, Br, CN, OMe, Ph, PhO, PhCH₂, NO₂, Me₂N, Me, EtO₂C, Ac, EtO, iPr, MeS, C₁-C₂₀ alkyl, C₁⁻C₂₀ heteroalkyl, C₁-C₁₀ alkyl-C₅-C₆ aryl, C₁-C₂₀ heteroalkyl-C₅-C₆ aryl.

The term heteroalkyl refers to an alkyl group, in which one or more carbon atoms are replaced by an oxygen, nitrogen, phosphorus or sulphur

Preferably n is 1 or 2, Ar is a phenyl or a phenyl substituted with a halogen in position 3.

Preferably the inhibitor of carbonic anhydrase is 4-(2-Aminoethyl)benzenesulfonamide.

Within the meaning of the present invention linker means a simple bond between the absorber of near infrared (NIR) light and the inhibitor of carbonic anhydrase or a compound of the general formula (II):

-HS-[(CH₂)ₙ-X-(CH₂)ₙ]-R- (II)

wherein
n is an integer from 1 to 4
X is NQ, O or S
R is NQH, OH, COOR′
R′is H, Me, Et,n-Pr, halogen
Q is H, C₁-C₂₀ alkyl, C₁-C₂₀ heteroalkyl, C₁-C₁₀ alkyl-C₅-C₆ aryl, C₁-C₂₀ heteroalkyl-C₅-C₆ aryl.

Preferably X is O, n is 2 or 3 and R is COOH.

More preferably X is O, n is 2 and R is COOH and the linker is alpha mercapto omega carboxy polyethylene glycol with a M. W. of 5000.

Optionally the absorber of near infrared (NIR) light is coated with a biocompatible polymer, preferably a biocompatible polymer with a molecular weight between 1000 and 10000 g mol⁻¹.

Within the meaning of the present invention biocompatible polymer means any polymer suitable for pharmaceutical use.

Preferably the biocompatible polymer is polyethylene glycol.

The assembly may optionally further comprise at least one drug or oligonucleotide bound to the absorber of near infrared (NIR) light by a cross-linker able to dissociate at a temperature between 40-100° C.

In this case the assembly releases the drug or oligonucleotide when subjected to near infrared light.

Preferably the cross-linker is either of:
a molecule covalently bound to the absorber of NIR light and the drug or oligonucleotide or
two molecules non-covalently bound to each other, where the one is covalently bound to the absorber of NIR light and the other to the drug or oligonucleotide.

More preferably said two molecules comprise a couple of complementary oligonucleotide strands undergoing hybridization below 40° C. and melting in a range of temperatures between 40-100° C.

Preferably the drug is selected from the group consisting of: alkylating agents, antimetabolites, anthracyclines, plant alkaloids, topoisomerase inhibitors, tyrosine kinase inhibitors, monoclonal antibodies and other antineoplastic agents.

Preferably the oligonucleotide is selected from the group consisting of: antisense, short interfering RNA, antigen, DNA decoy, RNA decoy, ribozyme and aptamer oligonucleotides.

Said assembly can be used as a contrast agent for photothermal treatments.

Said assembly can be used as a contrast agent for photoacoustic imaging.

A further object of the present invention is the use of said assembly as a medicament.

Another object of the present invention is the use of said assembly for use as an anticancer agent in hyperthermia therapy of tumours or conditions in which the CA activity is involved.

Within the meaning of the present invention tumours are: carcinomas, sarcomas, lymphomas, leukemias, germ cell tumours, blastomas, melanomas, brain tumours and nervous system tumours.

Within the meaning of the present invention, the conditions in which the CA activity is involved are for example cancer, tumour, glaucoma, obesity, osteoporosis, Alzheimer, epilepsy.

The assembly of the present invention may be prepared by a process comprising the following steps:
a) Contacting the NIR absorber with the linker and reacting to obtain a covalent bond between the NIR absorber and the linker;
b) Contacting the NIR absorber covalently bound to the linker as obtained in step a) with the CAI and reacting to obtain the assembly.

Or alternatively:
a) Contacting the CAI with the linker and reacting to obtain a covalent bond between the CAI and the linker;
b) Contacting the CAI covalently bound to the linker as obtained in step a) with the NIR absorber and reacting to obtain the assembly.

In a preferred embodiment wherein the near infrared absorber is a gold particle, the CAI displays an amino moiety and the cross linker contains mercapto and carboxyl terminations which bind the gold particle and amino moiety from the CAI by amidation.

In a preferred embodiment of the present invention the assembly comprises gold nanorods with effective radii (r=(3*V/(4*n))̂(1/3), with V particle volume) of ˜10 nm and an aspect ratio (AR=1/d, with 1 and d particle length and diameter respectively) of ˜4, which has an estimated optical absorption cross section of 4000 nm², conjugated with 4-(2-aminoethyl)benzenesulfonamide by means of an alpha mercapto omega carboxy polyethylene glycol cross linker binding the gold particle through a gold sulphur bridge and the amino termination from 4-(2-aminoethyl)benzenesulfonamide upon amidation.

In a further preferred embodiment the above gold nanorods are modified with a mixture of ˜90% alpha mercapto omega methoxy and 10% alpha mercapto omega carboxy polyethylene glycol (MW 5000 g mol⁻¹).

### EXAMPLES

Gold nanorods with effective radii of ˜10 nm and an aspect ratio of ˜4 may be conjugated with 4-(2-aminoethyl)benzenesulfonamide by the use of an alpha mercapto omega carboxy polyethylene glycol cross linker, binding the gold particle through a gold sulphur bridge and the amino termination from 4-(2-aminoethyl)benzenesulfonamide upon amidation.

Gold nanorods were synthesised at 25° C. in the dark. A seed suspension. was prepared by rapid. injection of ice cold 10 mM aqueous sodium borohydride into a solution containing 100 mM cetrimonium bromide and 240 μM chloroauric acid until a final concentration of 560 μM sodium borohydride. This suspension was left under vigorous agitation for 10 min and then at rest for another 2 hours before rapid addition into a growth solution. Meanwhile 500 ml growth. solution were prepared with 100 mM cetrimonium bromide, 470 μM chloroauric acid, 92 μM silver nitrate and 520 μM ascorbic acid, which was dosed with a burette in order to keep a precise ratio between gold ions and their reducing agents. This growth solution was injected with 1 ml seed suspension and left under mild agitation for 30 min and then at rest for 24 hours. Finally 100 mM aqueous ascorbic acid was added until a total content of 710 μM ascorbic acid, which served to complete the metal reduction, as it is described elsewhere (Ratto, J. Nanopart, Res. 12, 2029-2036 (2010)).

After two cycles of centrifugation and decantation (dead. volume ratio ˜1/200), these particles were transferred at a rate of 1.9 mM gold into an acetate buffer at pH 5 containing 500 μM cetrimonium bromide and 50 μM of a mixture of ˜90% alpha mercapto omega methoxy and 10% alpha mercapto omega carboxy polyethylene glycol (MW 5000 g mol⁻1). This suspension was left to react at 37° C. for two hours before centrifugation, decantation. and resuspension in 1% (v/v) aqueous polysorbate 20, which was left at rest for 2 hours at 37° C. in an attempt to extract possible contaminants.

After purification by two cycles of centrifugation and decantation, these particles were transferred at a rate of 1.9 mM gold into a MES buffer at pH 6 containing 130 mM NaCl and 0.005% (v/v) polysorbate 20. In a typical modification, 1.5 ml of this suspension were activated with 1.5 ml 16 mM 1-ethyl-3-(3-dimethylaminopropyl)carbodiimide and 4 mM N-hydroxysuccinimide, left at rest for 15 min, added with 3 ml 200 μM 4-(2-aminoethyl)henzenesulfonamide and then kept at rest for another 2 hours. Chemicals were dissolved in a MES buffer at pH 6 containing 130 mM NaCl and 0.005% (v/v) polysorbate 20 and maintained at 37° C. throughout the modification. Finally these particles were transferred at a rate of 4.0 mM gold into sterile PBS after four cycles of centrifugation and decantation.

The above assembly proves adequate to minimize the unspecific uptake of the particles by normoxic cells and enables an adequate load of the inhibitor by amidation. The prepared assemblies prove stable in physiological buffers and biological fluids and display irrelevant cytotoxicity up to 400 μM gold. Their cellular internalization may only occur via endocytotic pathways, i.e. these particles may not come into contact with intracellular carbonic anhydrases isoforms. Conversely their cellular uptake correlates with the expression of CA9, which may be modulated with oxygenation as it is documented in the scientific literature.

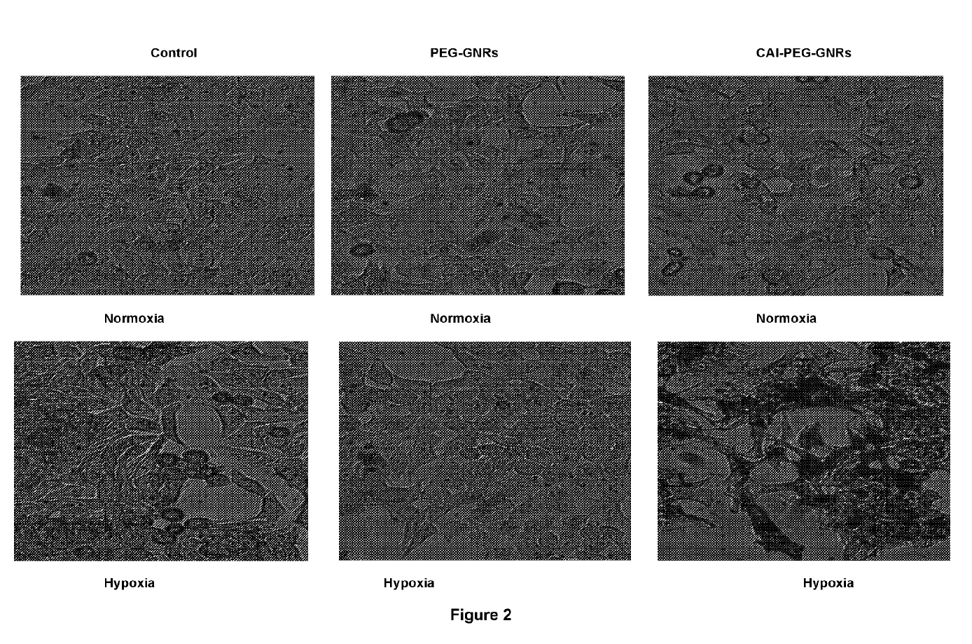

The results of targeting with inhibitors of CA9 are presented in FIG. 2 wherein the images display HCT116 colon carcinoma cells expressing transmembrane carbonic anhydrases only under hypoxic conditions. The accumulation of gold particles is revealed by silver enhancement only in the presence of the carbonic anhydrase inhibitor and under hypoxia. In turn the accumulation of these particles induces a substantial sensitization and decrease of the thresholds for cellular damage upon irradiation with NIR light under CW illumination. This effect already becomes visible in a cellular monolayer despite its inefficient heat confinement.

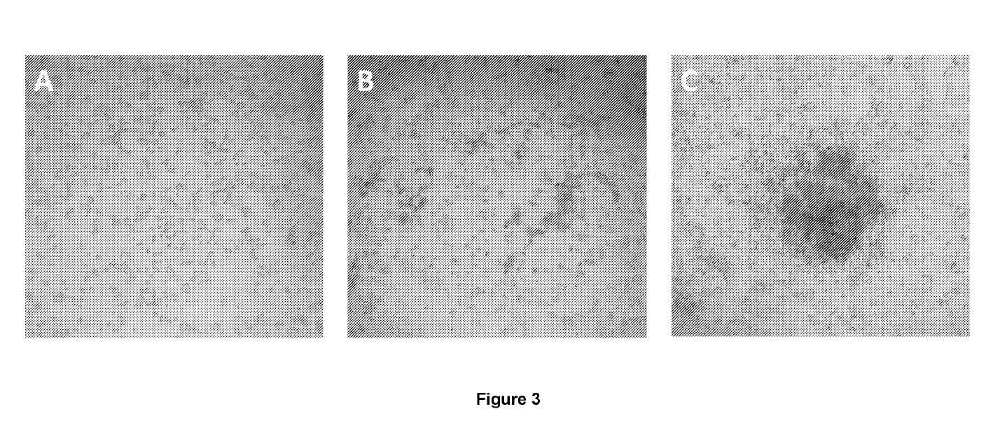

FIG. 3 reports images showing HCT116 colon carcinoma cells maintained under hypoxic conditions and then treated for ten minutes with a NIR laser diode with a power density of 60 W m². Panels display cells without particles (A) and incubated with PEGylated particles without (B) and with ligand (C). After incubation, the cellular medium was thoroughly replaced. The synergistic effect of the inhibitor and optical excitation leads to cell death, which is identified by trypan blue staining in panel C. In the absence of ligand this optical fluence does not damage cells. The power density required to reach the damage threshold both under hypoxia with particles without ligands and normoxia with particles with ligands falls between around 120-150 W cm².

1. Assembly comprising an absorber of near infrared (NIR) light having an optical absorption cross section not lower than 100 nm² and an inhibitor of carbonic anhydrase covalently bound by a linker.

2. Assembly according to claim 1 wherein the absorber of near infrared (NIR) light has an optical absorption cross section ranging from 102 to 105 nm².

3. Assembly according to claim 1 wherein the absorber of near infrared (NIR) light has an optical absorption cross section ranging from 103 to 104 nm².

4. Assembly according to claim 1, wherein the absorber of near infrared (NIR) light is selected from the group consisting of: nanoparticles containing near infrared dyes (organic compounds with conjugated n systems containing polyene, polymethine or donor-acceptor near infrared chromophores, including indocyanine green, IR-780 iodide) within solid lipid, liposomal, micellar, porous silica, polymeric or viral carriers; phospholipid porphyrin conjugates; organic nanoparticles comprising single walled carbon nanotubes, multi walled carbon nanotubes, graphene, graphene oxide; metallic nanoparticles with near infrared plasmonic resonances comprising gold nanorods, gold/silica nanoshells, gold nanocages, gold/gold sulfide nanoshells, hollow gold nanospheres; up-conversion nanoparticles containing lanthanide moieties, and combinations thereof.

5. Assembly according to claim 4 wherein the absorber of near infrared (NIR) light is a plasmonic particle.

6. Assembly according to claim 5 wherein the plasmonic particle has a size between ˜5 and 100 nm.

7. Assembly according to claim 5 wherein the plasmonic particle is a gold nanorod.

8. Assembly according to claim 1, wherein the inhibitor of carbonic anhydrase is an inhibitor of carbonic anhydrase 9 (CA9).

9. Assembly according to claim 1, wherein the inhibitor of carbonic anhydrase is the compound of general formula (I):
X-(CH2)n-Ar-[Q]-SO2NH2
wherein
n is an integer from 0 to 2
Q is absent or is O or N
X is N, O, S or COOR
R is H, Me, Et, n-Pr, halogen
Ar is an aromatic or heteroaromatic ring optionally substituted with at least one substituent selected from the group consisting of: Cl, F, I, Br, CN, OMe, Ph, PhO, PhCH₂, NO₂, Me₂N, Me, EtO₂C, Ac, EtO, iPr, MeS, C₁-C₂₀ alkyl, C₁-C₂₀ heteroalkyl, C₁-C₁₀ alkyl-C₅-C₆ aryl, C₁-C₂₀ heteroalkyl-C₅-C₆ aryl.

10. Assembly according to claim 9 wherein in general formula (I) n is 1 or 2, Ar is a phenyl or a phenyl substituted with a halogen in position 3.

11. Assembly according to claim 9 wherein the inhibitor of carbonic anhydrase is 4-(2-Aminoethyl)benzenesulfonamide.

12. Assembly according to claim 1, wherein the linker is a simple bond between the absorber of near infrared (NIR) light and the inhibitor of carbonic anhydrase or a compound of general formula (II):
—HS—[(CH2)n—X—(CH2)n]—R—
wherein
n is an integer from 1 to 4
X is NQ, O or S
R is NQH, OH, COOR′
R′is H, Me, Et,n-Pr, halogen
Q is H, C₁-C₂₀ alkyl, C₁-C₂₀ heteroalkyl, C₁-C₁₀ alkyl-C₅-C₆ aryl, C₁-C₂₀ heteroalkyl-C₅-C₆ aryl.

13. Assembly according to claim 12 wherein in general formula (II) X is O, n is 2 or 3 and R is COOH.

14. Assembly according to claim 13 wherein in general formula (II) X is O, n is 2 and R is COOH.

15. Assembly according to claim 14 wherein the linker is alpha mercapto omega carboxy polyethylene glycol with a M.W. of 5000.

16. Assembly according to claim 1, wherein the absorber of near infrared (NIR) light is coated with a biocompatible polymer.

17. Assembly according to claim 16 wherein the biocompatible polymer has a molecular weight between 1000 and 10000 g mol⁻¹.

18. Assembly according to claim 17 wherein the biocompatible polymer is polyethylene glycol.

19. Assembly according to claim 1, wherein gold nanorods with effective radii of ˜10 nm and an aspect ratio of ˜4 are linked to 4-(2-aminoethyl)benzenesulfonamide by means of an alpha mercapto omega carboxy polyethylene glycol cross linker.

20. Assembly according to claim 1 further comprising at least one drug or oligonucleotide bound to the absorber of near infrared (NIR) light by a cross-linker able to dissociate at a temperature between 40-100° C.

21. Assembly according to claim 1 for use as contrast agent for photothermal treatments.

22. Assembly according to claim 1 for use as a contrast agent for photoacoustic imaging.

23. Assembly according to claim 1 for use as a medicament.

24. Assembly according to claim 1 for use as anticancer agent in hyperthermia therapy of tumours or conditions in which the carbonic anhydrase (CA) activity is involved.

25. Assembly according to claim 1 for use as carrier for photothermal release of drugs and oligonucleotides.

26. Use according to claim 24 wherein the condition in which the CA activity is involved is selected from the group consisting of: cancer, tumour, glaucoma, obesity, osteoporosis, Alzheimer, and epilepsy.

27. Process for the preparation of the assembly of claim 1 comprising the following steps:
a) contacting the NIR absorber with the linker and reacting to obtain a covalent bond between the NIR absorber and the linker;
b) contacting the NIR absorber covalently bound to the linker as obtained in step a) with the carbonic anhydrase inhibitor (CAI) and reacting to obtain the assembly.
or alternatively:
a) contacting the CAI with the linker and reacting to obtain a covalent bond between the CAI and the linker;
b) contacting the CAI covalently bound to the linker as obtained in step a) with the NIR absorber and reacting to obtain the assembly. 28) (Original) Process according to claim 27 wherein gold nanorods with effective radii of ˜10 nm and an aspect ratio of ˜4 are linked to 4-(2-aminoethyl)benzenesulfonamide by means of an alpha mercapto omega carboxy polyethylene glycol cross linker through a gold sulphur bridge and the amino termination from 4-(2-aminoethyl)benzenesulfonamide upon amidation.
